(19) United States
(12) Patent Application Publication
(10) Pub. No.: US 2009/0263481 A1
(43) Pub. Date: Oct. 22, 2009

(54) LEVETIRACETAM FORMULATIONS

(76) Inventors: Atul Vishvanath Patil, Belgaum (IN); Narayanan Badri Vishwanathan, Chennai (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/425,051

(22) Filed: Apr. 16, 2009

Related U.S. Application Data
(60) Provisional application No. 61/081,534, filed on Jul. 17, 2008.

(30) Foreign Application Priority Data
Apr. 17, 2008 (IN) 962/CHE/2008

(51) Int. Cl. A61K 9/24; A61K 31/4015; A61P 25/08

(52) U.S. Cl. 424/472; 514/424

(57) ABSTRACT

Pharmaceutical formulations comprising levetiracetam and having an inner solid phase and outer continuous phase, wherein one or both of the phases comprise at least one hydrophobic material, lipophilic material, or combination thereof.

The present invention relates to pharmaceutical formulations comprising levetiracetam or any of its derivatives. Further the formulations relate to pharmaceutical formulations comprising levetiracetam or any of its derivatives, wherein the formulations comprise a hydrophobic core material. The invention also relates to processes for preparing the formulations and methods of using the formulations in treating seizures.

Levetiracetam is an S-enantiomer of etiracetam. Levetiracetam has a chemical name (−)-(S)-α-ethyl-2-oxo-1-pyrrolidine acetamide. It has the chemical formula C₈H₁₄N₂O₂ that corresponds to a molecular weight of 170.21, and it is represented by structural formula (1).

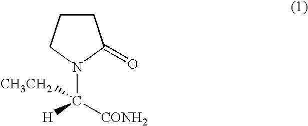

Levetiracetam is a white to off white crystalline powder and is very soluble in water with an aqueous solubility of 104 g/100 mL, is freely soluble in chloroform, methanol, and is soluble in ethanol. The drug is sparingly soluble in acetonitrile and practically insoluble in n-hexane. It is a muscarinic receptor agonist.

Levetiracetam is indicated as adjunctive therapy in the treatment of partial onset seizures in adults with epilepsy.

Levetiracetam is available in four dosage forms: immediate release tablets; an oral solution; an injectable infusion; and extended release tablets. Levetiracetam is commercially available and is approved for sale in various countries including the United States of America under the brand name KEPPRA™ and KEPPRA XR™ from UCB Pharma. KEPPRA is available in 250, 500, 750 and 1000 mg strengths as immediate release tablet formulations. Inactive excipients for these tablets include colloidal silicon dioxide, croscarmellose sodium, magnesium stearate, polyethylene glycol 3350, polyethylene glycol 6000, polyvinyl alcohol, talc, titanium dioxide and colouring agents.

Levetiracetam is also available as KEPPRA oral solution with the strength of 100 mg/ml. Inactive excipients include ammonium glycyrrhizinate, citric acid monohydrate, glycerin, maltitol solution, methyl paraben, potassium acesulfame, propylparaben, purified water, sodium citrate dihydrate and natural and artificial flavor. Also available is KEPPRA injectable solution of 100 mg/ml strength, wherein inactive excipients include water for injection, sodium chloride buffered at pH 5.5 using glacial acetic acid and 8.2 mg sodium acetate trihydrate.

Levetiracetam is rapidly and almost completely absorbed after oral administration. Absorption of levetiracetam is rapid, with peak plasma concentrations occurring within about one hour following oral administration to fasted subjects. The oral bioavailability of levetiracetam tablets is 100% and the tablets and oral solution are bioequivalent in their rate and extent of absorption. Food does not affect the extent of absorption of levetiracetam, but it decreases Cₘₐₓ by 20% and delays Tₘₐₓ by 1.5 hours. The pharmacokinetics of levetiracetam are linear over the dose range of 500-5000 mg. Steady state is achieved after 2 days of twice-daily dosing. Levetiracetam and its major metabolite are less than 10% bound to plasma proteins; clinically significant interactions with other drugs through competition for protein binding sites are therefore unlikely.

Levetiracetam is also available in extended release form as KEPPRA XR™ with the strength of 500 mg. Inactive ingredients include colloidal anhydrous silica, hypromellose, magnesium stearate, polyethylene glycol 6000, polyvinyl alcohol-partially hydrolyzed, titanium dioxide, macrogol/PEG 3350, and talc. The recommended initial dose is 1000 mg once daily, with gradual adjustment to a maximum of 3000 mg daily.

Levetiracetam is described in U.S. Pat. Nos. 4,837,223, 4,943,639, and 6,107,492.

International Application Publication Nos. WO 2006/080029, WO 2006/088864, WO 2006/123357, WO 2008/006528, WO 2008/062446, and WO 2008/113901, and United States Patent Application Publication Nos. 2006/0165796, 2007/0092569, 2007/0298098, 2008/0014264, and 2008/0014271, describe formulations of levetiracetam.

KEPPRA products are administered twice daily with or without food administration. Major side effects include somnolence, asthenia, headache, infection, pain, dizziness. There is need for controlling the release of drug substance from the formulations and/or formulations so that they can be administered in fewer daily doses, such as once daily administration. Also, since it is a centrally acting drug substance, there has been a need to reduce the above discussed side effects.

Levetiracetam, being a highly soluble and high dose drug substance, poses a challenge to the formulator for developing controlled release rate dosage forms. When large quanitities of excipients are used to control the release, then dosage form sizes will be high, making oral administration problematic.

### SUMMARY

The present invention relates to pharmaceutical compositons comprising levetiracetam or its derivatives. Also, the invention relates to processes of preparing formulations and methods of using such formulations for treating seizures.

In an embodiment, the invention includes pharmaceutical formulations comprising levetiracetam and at least one hydrophobic material in an inner solid phase.

In an embodiment, the invention includes pharmaceutical composiitons comprising levetiracetam and at least one lipophilic material in an inner solid phase.

In an embodiment, the invention includes pharmaceutical formulations comprising levetiracetam and a combination of hydrophobic and lipophilic materials in an inner solid phase.

In embodiments, the invention includes pharmaceutical compositons comprising levetiracetam and at least one hydrophobic and/or lipophilic material wherein weight ratios of levetiracetam to hydrophobic and/or lipophilic materials are in the range of about 1:0.001 to about 1:10, or 1:0.001 to about 1:5, or about 1:0.1 to about 1:1

In embodiments, the invention includes pharmaceutical formulations comprising a pharmacologically inert inner phase and an outer continuous phase comprising levetiracetam and at least one hydrophobic material.

In embodiments, the invention includes pharmaceutical formulations comprising a pharmacologically inert inner phase and an outer continuous phase comprising levetiracetam and at least one lipophilic material.

In embodiments, the invention includes pharmaceutical formulations comprising a pharmacologically inert inner phase and an outer continuous phase comprising levetiracetam and combinations of at least one each from hydrophobic and lipophillic materials.

In embodiments, the invention includes pharmaceutical formulations comprising a pharmacologically inert inner phase and a continuous phase wherein levetiracetam is in intimate contact with hydrophobic and/or lipophillic materials.

In embodiments, the invention includes pharmaceutical formulations wherein both an inner solid phase and an outer continuous phase comprise levetiracetam in combination with at least one hydrophobic or lipophillic material, or a combination thereof.

In embodiments, the invention includes pharmaceutical formulations wherein an inner solid phase comprises levetiracetam optionally with at least one pharmaceutical excipient and an outer continuous phase comprises hydrophobic, lipophilic, or hydrophilic materials.

In embodiments, the invention includes pharmaceutical formulations comprising levetiracetam, in the form of controlled release formulations providing once-daily administration.

### DETAILED DESCRIPTION

The present invention relates to pharmaceutical compositons comprising levetiracetam, including any of its derivatives. Also, the invention relates to processes for preparing formulations and methods of using such formulations for treating seizures.

Due to the high degree of bioavailability and rapid metabolism of levetiracetam, it would be advantageous to provide controlled release levetiracetam with a drug-delivery formulation that releases the active ingredient with a controlled, delayed, or extended release profile. More specifically, it would be a tremendous benefit to patients suffering from epilepsy if the drug could be formulated to be released in a controlled manner, so that the drug can provide its pharmacological activity over an extended period of time, in particular, a twenty-four hour period, instead of being rapidly removed by metabolism. In this manner, patients suffering from epilepsy could benefit from the drug's therapeutic effects for extended periods of time.

The term “derivatives” for purposes of the present invention includes, without limitation thereto, salts, ethers, esters, prodrugs, enatiomers, isomers, various polymorphic forms, coprecipitates, etc., of leviteracetam.

The term “controlled release” for purposes of the present invention includes delayed release, extended release, modified release, and sustained release. It refers to dosage forms that release the contained drug after a time delay following administration, and/or at a predetermined rate that is slower than the rate obtained by an immediate release formulation.

In general, formulations of the present invention will release no more than about 50% of the contained drug within about the first 2 hours, no more than about 75% of the contained drug within about the first 4 hours, and no less than about 80% of the contained drug within about the first 12 hours, following immersion into an aqueous environment.

The term “inner solid phase” for purposes of the present invention includes a core, which may comprise drug substance alone, or drug substance in combination with hydrophobic or lipophilic excipients, or may be an inert particulate material, of a dosage form that may be presented as a tablet or minitablet, pellets, beads, granules, etc.

The term “outer continuous phase” for purposes of the present invention is a phase, which completely covers an inner solid phase.

The term “hydrophobic material” for purposes of the present invention relates to excipients, which are insoluble in water, which are water repellent, or which lack affinity toward water.

The term “lipophilic material” for purposes of the present invention relates to excipients which have strong affinity for oils and fats, or are soluble therein.

The term “pharmaceutical formulation” for purposes of the present invention includes matrix formulations, reservoir formulations, multiparticulate formulations, multilayer formulations, resin formulations, osmotic formulations, and gastro-retentive formulations.

The term “dosage form” for purposes of the present invention includes any solid oral pharmaceutical product, in a form such as tablets, capsules, sachets, pills, or granules.

In an embodiment the invention includes pharmaceutical formulations having two phases, an inner solid phase and an outer continuous phase.

In an embodiment the invention includes pharmaceutical formulations comprising levetiracetam and at least one hydrophobic material in an inner solid phase.

In another embodiment the invention includes pharmaceutical formulations comprising levetiracetam and at least one lipophilic material in an inner solid phase.

In yet another embodiment the invention includes pharmaceutical formulations comprising levetiracetam and a combination of at least one each from hydrophobic materials and lipophilic materials, in an inner solid phase.

In an embodiment the invention includes pharmaceutical formulations comprising levetiracetam in the form of controlled release formulations.

In an embodiment the invention includes hydrophobic or lipophilic materials or combinations thereof in intimate contact with levetiracetam in formulations, so that levetiracetam release is controlled over a predetermined period of time.

Levetiracetam in the formulations may be either in an inner solid phase or an outer continuous phase, or in both.

In an embodiment the invention includes a plurality of particles comprising an inert inner phase and an outer continuous phase comprising levetiracetam and at least one hydrophobic material.

In an embodiment the invention includes a plurality of particles comprising an inert inner phase and an outer continuous phase comprising levetiracetam and at least one lipophilic material.

In an embodiment the invention includes a plurality of particles comprising an inert inner phase and an outer continuous phase comprising levetiracetam and a combination of at least one each from hydrophobic and lipophilic materials.

In a further embodiment the invention includes a plurality of particles comprising an inert inner phase and a continuous phase wherein levetiracetam is in intimate contact with hydrophobic and/or lipophilic materials.

Hydrophobic materials that are suitable for use in the present invention include, but are not limited to, hydrophobic polymers which may be employed in the inner solid phase and/or outer continuous phase such as, but not limited to, ethylcelluloses, hydroxyethyl celluloses, ammoniomethacrylate copolymers (EUDRAGIT™ RL or EUDRAGIT RS or EUDRAGIT RL PO or EUDRAGIT RS PO or EUDRAGIT RD), methacrylic acid copolymers (EUDRAGIT L or EUDRAGIT S), methacrylic acid-acrylic acid ethyl ester copolymers (EUDRAGIT L 100-5), methacrylic acid esters neutral copolymers (EUDRAGIT NE 30D), dimethylaminoethylmethacrylate-methacrylic acid esters copolymers (EUDRAGIT E 100), and vinyl methyl ether/maleic anhydride copolymers, their salts and esters (GANTREZ™).

In an embodiment the invention includes pharmaceutical formulations comprising levetiracetam and a hydrophobic material comprising any one or more of ethylcellulose, EUDRAGIT RL, EUDRAGIT RS, EUDRAGIT RL PO, EUDRAGIT RS PO, EUDRAGIT RD, EUDRAGIT L, EUDRAGIT S, EUDRAGIT L 100-5, EUDRAGIT NE 30D, EUDRAGIT E 100.

EUDRAGIT polymers are products of Evonik Industries AG, Essen, Germany. GANTREZ polymers are products of International Specialty Products, Parsippany, N.J. USA.

Polymethacrylates are synthetic cationic and anionic polymers of dimethylaminoethyl methacrylates, methacrylic acid and methacrylic acid esters in varying molar ratios. Several different types are commercially available and may be purchased as dry powders, or in aqueous mixtures.

Polymers sold as EUDRAGIT have the following general repeating unit,

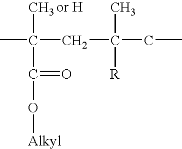

where R is COOH for the EUDRAGIT L products, R is COOCH₂N(CH₃)₂ for the EUDRAGIT E products, R is COOCH₃ for the EUDRAGIT NE 30 D product, and R is COOCH₂CH₂N⁺ (CH₃)₃Cl⁻ for the EUDRAGIT E and EUDRAGIT RS products. The alkyl groups vary between different products and have 1-4 carbons.

The United States Pharmacopoeia and National Formulary describes methacrylic acid copolymer as a fully polymerized copolymer of methacrylic acid and an acrylic or methacrylic ester. Three types of copolymers, namely Type A, Type B, and Type C, are defined in the monograph. They vary in their methacrylic acid content and solution viscosity. Type C may contain suitable surface-active agents. Two additional polymers, Type A (EUDRAGIT RL) and Type B (EUDRAGIT RS), also referred to as ammonio methacrylate copolymers, consisting of fully polymerized copolymers of acrylic and methacrylic acid esters with a low content of quaternary ammonium groups, are also described.

Lipophilic materials that are suitable for use in the present invention include, but are not limited to: waxes such as beeswax, carnauba wax, microcrystalline wax, and ozokerite; fatty alcohols such as cetostearyl alcohol, stearyl alcohol, cetyl alcohol and myristyl alcohol; and fatty acid esters such as glyceryl monostearate, glycerol monooleate, acetylated monoglycerides, stearin, palmitin, laurin, myristin, cetyl esters wax, glyceryl palmitostearate, glyceryl behenate, hydrogenated castor oil, and cottonseed oils.

In an embodiment the invention includes pharmaceutical formulations comprising levetiracetam, wherein concentrations of hydrophobic and/or lipophilic materials are in the ranges of about 1% to about 50%, or about 1% to about 25%, by weight.

In an embodiment, the invention includes pharmaceutical tablets comprising about 10% to about 75% of levetiracetam, and from about 1% to about 50%, or about 1% to about 25%, of hydrophobic and/or lipophilic materials, by weight.

A pharmacologically inert inner phase for use in the present invention may have regular or irregular shapes, such as spherical or non-spherical. In one of the embodiments the invention includes the inert inner phase in a substantially spherical shape. An inert inner phase may be an inert particle or placebo tablet.

Useful inert inner phase materials include, but are not limited to: water-insoluble inert materials, such as glass particles or silicon dioxide, calcium phosphate dihydrate, dicalcium phosphate, calcium sulfate dihydrate, microcrystalline celluloses (including Celphere™ microcrystalline cellulose spheres sold by Asahi Kasei Chemicals Corporation, Tokyo, Japan), cellulose derivatives; soluble inert materials such as sugar spheres having sugars like sucrose, dextrose, lactose, anhydrous lactose, spray-dried lactose, lactose monohydrate, or mannitol, starches, sorbitol; insoluble inert plastic materials, i.e., spherical or nearly spherical core beads of polyvinylchloride, polystyrene or any other pharmaceutically acceptable insoluble synthetic polymeric material, and the like; and mixtures thereof.

In an embodiment the invention includes pharmaceutical formulations wherein the inner solid phase comprises levetiracetam and optionally at least one pharmaceutically acceptable excipient, and an outer continuous phase comprises one or more hydrophobic, lipophilic, or hydrophilic materials, and wherein the release is controlled by an outer continous phase.

Useful hydrophilic materials for the present invention include but are not limited to: cellulose derivatives such as hydroxypropyl methylcelluloses such as METHOCEL™ K or E, hydroxyethyl celluloses, hydroxypropyl celluloses, methylcelluloses and the like; noncellulose polysaccharides including galactomannans, guar gum, carob gum, gum arabic, sterculia gum, agar, alginates, xanthum gum and the like; polyvinylpyrrolidones; polyvinylacetates; mixtures of polyvinylpyrrolidone and polyvinylacetate, such as KOLLIDON™ SR; and acrylic acid polymers like crosslinked acrylic acid-based polymers such as a Carbopol™.

In an embodiment the invention includes an outer continuous phase in the form of a coating over the inner solid phase.

An outer continuous phase may comprise either hydrophobic or lipophilic materials as discussed above, and may be combined with levetiracetam.

In embodiments the invention includes pharmaceutical compositons comprising levetiracetam and at least one hydrophobic and/or lipophilic material in the core, wherein weight ratios of levetiracetam to hydrophobic and/or lipophilic material is in the range of about 1:0.001 to about 1:1, or about 1:0.1 to about 1:0.5.

In an embodiment the invention includes pharmaceutical formulations comprising levetiracetam and at least one hydrophobic and/or lipophilic material, wherein a concentration of hydrophobic and/or lipophilic material is in the range of about 1% to about 50%, or about 1% to about 25%, by weight.

The pharmaceutical formulations of the present invention may be prepared using any techniques such as dry granulation, wet granulation, direct compression, melt granulation, extrusion-spheronization, etc., including combinations thereof.

The pharmaceutical formulations of the present invention may be further formulated into dosage forms such as tablets, capsules, sachets, pills, or granules.

Apart from the above discussed functional excipients such as hydrophobic materials and lipophilic materials the dosage forms can further comprise various other pharmaceutically acceptable excipients such as any one or more of diluents, binders, disintegrants, glidants, lubricants, colouring agents, and coating materials.

Diluents include but are not limited to starches, lactose, mannitol, Pearlitol™ SD 200, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline celluloses and powdered celluloses. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, directly compressible grade of dibasic calcium phosphate (Emcompress), and tribasic calcium phosphate.

Binders include but are not limited to hydroxypropylcelluloses (Klucel™ LF), hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcellulose, polymethacrylates, and starch.

Disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcelluloses include but are not limited to grades such as LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

Solvents that are useful in processing include, but are not limited to, water, methanol, ethanol, isopropanol, butanols, acidified ethanol, acetone, diacetone, polyols, polyethers, oils, esters, alkyl ketones, methylene chloride, methyl acetate, ethyl acetate, isopropyl acetate, castor oil, ethylene glycol monoethyl ether, diethylene glycol monobutyl ether, diethylene glycol monoethyl ether, dimethylsulphoxide, dimethylformamide, tetrahydrofuran, and mixtures thereof.

Glidants or antisticking agents can be used, including but not limited to talc, silica derivatives, colloidal silicon dioxide and the like or mixtures thereof, and lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes.

Various useful colourants include but are not limited to Food Yellow No. 5, Food Red No. 2, Food Blue No. 2, and the like, food lake colorants, and iron oxides.

If desired, an outer continuous phase in the form of a film may be used, optionally contain additional adjuvants for coating processing such as plasticizers, polishing agents, colorants, pigments, antifoam agents, opacifiers, antisticking agents, and the like.

Various plasticizers include but are not limited to castor oil, diacetylated monoglycerides, dibutyl sebacate, diethyl phthalate, glycerin, polyethylene glycol, propylene glycol, triacetin, triethyl citrate. Also mixtures of plasticizers may be utilized. The type of plasticizer depends upon the type of coating agent. A plasticizer is frequently present in an amount ranging from 5% (w/w) to 30 (w/w) based on the total weight of the film coating.

An opacifier like titianium dioxide may also be present in an amount ranging from about 10% (w/w) to about 20% (w/w) based on the total weight of the coating. When coloured tablets are desired then the colour is normally applied in the coating. Consequently, colouring agents and pigments may be present in the film coating. Various colouring agents include but not limited to iron oxides, which can be red, yellow, black or blends thereof.

Anti-adhesives are frequently used in the film coating process to avoid sticking effects during film formation and drying. An example of an anti-adhesive for this purpose is talc. The anti-adhesive typically is present in the film coating in an amount of about 5% (w/w) to 15% (w/w) based upon the total weight of the coating.

Suitable polishing agents include polyethylene glycols of differing molecular weights or mixtures thereof, talc, surfactants (e.g., glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In some embodiments, polyethylene glycols having molecular weight of 3,000-20,000 are employed.

As alternatives for the above coating ingredients, sometimes pre-formulated coating products such as those sold as OPADRY™ (supplied by Colorcon) will be used, for example Opadry Blue 13B50579 or Opadry White 04-58900. The products sold in a solid form require only mixing with a liquid before use.

Equipment suitable for processing the pharmaceutical formulation of the present invention includes any one or a combination of mechanical sifters, blenders, roller compactors, granulators (rapid mixer or fluid bed granulator) fluid bed dryers, compression machines, rotating bowls or coating pans, etc.

The present invention further relates to processes for manufacturing pharmaceutical formulations of the present invention, wherein an embodiment of a process comprises:

1 ) Sifting drug substance, hydrophobic or lipophilic materials and other excipients such as diluents, disintegrants etc through a sieve and mixing.

2) Granulating step 1) materials using solvent or granulating fluid.

3) Drying the granules.

4) Sifting the dried granules and extragranular excipients through a sieve.

5) Blending sifted granules and extragranular excipients and adding a lubricant to the blend.

6) Compressing the final lubricated blend into tablets or filling into capsules.

7) Coating tablets or capsules with hydrophobic, lipophilic, or hydrophilic materials along with other coating adjuvants.

Alternatively, step 1) materials can be blended with extragranular excipients and lubricants and compressed into tablets or filled into capsules. As another alternative, step 1) materials may be compacted and milled through a screen, to form granules that can be blended with extragranular materials and lubricants and compressed into tablets or filled into capsules.

The tablets or capsules prepared as above can be subjected to in vitro dissolution evaluation according to Test 711 “Dissolution” in United States Pharmacopoeia 29, United States Pharmacopeial Convention, Inc., Rockville, Md., 2005 (“USP”) to determine the rate at which the active substance is released from the dosage forms, and content of active ingredient can conveniently be determined in solutions by techniques such as high performance liquid chromatography.

In embodiments, the invention includes the use of packaging materials such as containers and closures of high-density polyethylene (HDPE), low-density polyethylene (LDPE) and/or polypropylene and/or glass, and blisters or strips composed of aluminum, high-density polypropylene, polyvinyl chloride, polyvinylidine dichloride, etc.

Certain specific aspects and embodiments of the invention will be further described in the following examples, which are provided only for purposes of illustration and are not intended to limit the scope of the invention in any manner.

### EXAMPLE 1

### Levetiracetam 500 mg Extended Release Tablets

| | |
|---|---|
| Ingredient | Grams |

Tablet Core

| | |
|---|---|
| Levetiracetam | 100 |
| EUDRAGIT ™ RS PO # | 27 |
| EUDRAGIT RL PO $ | 3 |
| Microcrystalline cellulose PH102 | 20 |
| Magnesium stearate | 0.75 |
| Acetone* | 30 |
| Microcrystalline cellulose PH102 | 10 |
| Talc | 1.5 |
| Colloidal silicon dioxide | 1.5 |
| Magnesium stearate | 0.75 |

Extend Release Coating

| | |
|---|---|
| Ethylcellulose 7 cps | 15 |
| EUDRAGIT RL PO | 15 |
| Triethyl citrate | 3 |
| Isopropyl alcohol- | 300 |
| dichloromethane (1:1 by volume)* | |

*Evaporates during processing.
# EUDRAGIT RS PO chemically is poly(ethylacrylate, methylmethacrylate, trimethyl ammonioethyl methacrylate chloride) 1:2:0.1, supplied by Evonic Industries, Germany.
$ EUDRAGIT RL PO chemically is poly(ethylacrylate, methylmethacrylate, trimethyl ammonioethyl methacrylate chloride) 1:2:0.2, supplied by Evonic Industries, Germany.

Manufacturing process:

A. Tablet Core:

1) Levetiracetam, EUDRAGIT RLPO, EUDRAGIT RSPO, microcrystalline cellulose (first quantity), and magnesium stearate were sifted through an ASTM #40 mesh sieve.

2) Step 1) materials were dry mixed and granulated with acetone.

3) Granules were dried at an inlet temperature of 60° C. and sifted through an ASTM #30 mesh sieve.

4) Microcrystalline cellulose (second quantity) was sifted through an ASTM #40 mesh sieve and talc, colloidal silicon dioxide and magnesium stearate were sifted through an ASTM #60 mesh sieve.

5) Granules of step 3) were blended with sifted excipients of step 4) for about 5 minutes.

6) Granules of step 5) were compressed into tablets with hardness ranging from about 12 to 15 Kp.

B. Extended Release Coating:

1) The compressed tablets prepared above were placed in a perforated coating plan and preheated with an inlet temperature of 45° C.

2) Extended release coating solution was prepared by dissolving ethylcellulose and EUDRAGIT RL PO in an isopropyl alcohol-dichloromethane mixture with the addition of the plasticizer triethyl citrate.

3) Tablets were coated with an inlet temperature of 45° C. with atomization of 1.0 bar. The coated tablets were removed at regular intervals according to their weight gain after drying: 2% (1A), 4% (1B) and 6% (1C).

The coated tablets were tested for dissolution characteristics in USP apparatus 2, 900 ml of water, at 100 RPM stirring. The data are tabulated in Table 1.

TABLE 1

| | Cumulative % | | |
|---|---|---|---|
| | of Drug Dissolved | | |
| Hours | 1A | 1B | 1C |
| 1 | 24 | 12 | 7 |
| 2 | 41 | 27 | 20 |
| 4 | 64 | 49 | 42 |
| 8 | 79 | 64 | 57 |
| 12 | 96 | 89 | 83 |

### EXAMPLE 2

### Levetiracetam 500 mg Extended Release Tablets

| | |
|---|---|
| Ingredient | Grams |

Tablet Core

| | |
|---|---|
| Levetiracetam | 100 |
| EUDRAGIT RS PO | 27 |
| EUDRAGIT RL PO | 3 |
| Microcrystalline cellulose PH102 | 20 |
| Magnesium stearate | 0.75 |
| Acetone* | 30 |
| Microcrystalline cellulose PH102 | 10 |
| Talc | 1.5 |
| Colloidal silicon dioxide | 1.5 |
| Magnesium stearate | 0.75 |

Extended Release Coating

| | |
|---|---|
| Ethylcellulose 7 cps | 27 |
| EUDRAGIT RL PO | 3 |
| Triethyl citrate | 3 |
| Isopropyl alcohol- | 300 |
| dichloromethane (1:1 by volume)* | |

*Evaporates during processing.

Manufacturing process: similar to that of Example 1.

### EXAMPLE 3

### Levetiracetam 500 mg Extended Release Tablets with Water-soluble Filler

| | |
|---|---|
| Ingredient | Grams |

Tablet Core

| | |
|---|---|
| Levetiracetam | 100 |
| Lactose monohydrate | 60 |
| Isopropyl alcohol* | 30 |
| Talc | 1.5 |
| Colloidal silicon dioxide | 1.5 |
| Magnesium stearate | 0.75 |

Extended Release Coating

| | |
|---|---|
| Ethylcellulose 7 cps | 15 |
| EUDRAGIT RL PO | 15 |
| Triethyl citrate | 3 |
| Isopropyl alcohol-dichloromethane (1:1 | 300 |
| by volume)* | |

*Evaporates during processing.

Manufacturing process: similar to that of Example 1, except for the following differences:

1) EUDRAGIT RL PO, EUDRAGIT RS PO, and microcrystalline cellulose of step 1) were replaced with lactose monohydrate.

2) In step 2), granulation with isopropyl alcohol.

3) Microcrystalline cellulose was omitted from step 4).

### EXAMPLE 4

### Levetiracetam 500 mg Extended Release Tablets with Water-insoluble Swellable Filler

| | |
|---|---|
| Ingredient | Grams |

Tablet Core

| | |
|---|---|
| Levetiracetam | 100 |
| Microcrystalline cellulose PH102 | 60 |
| Isopropyl alcohol* | 30 |
| Talc | 1.5 |
| Colloidal silicon dioxide | 1.5 |
| Magnesium stearate | 0.75 |

Extended Release Coating

| | |
|---|---|
| Ethylcellulose 7 cps | 15 |
| EUDRAGIT RL PO | 15 |
| Triethyl citrate | 3 |
| Isopropyl alcohol-dichloromethane (1:1 | 300 |
| by volume)* | |

*Evaporates during processing.

Manufacturing process: similar to that of Example 3, except that lactose monohydrate was replaced by microcrystalline cellulose.

### EXAMPLE 5

### Levetiracetam500 mg Extended Release Tablets with Water-insoluble Non-swellable Filler

| | |
|---|---|
| Ingredient | Grams |

Tablet Core

| | |
|---|---|
| Levetiracetam | 100 |
| Dibasic calcium phosphate | 60 |
| (EMCOMPRESS ™) | |
| Isopropyl alcohol* | 30 |
| Talc | 1.5 |
| Colloidal silicon dioxide | 1.5 |
| Magnesium stearate | 0.75 |

Extended Release Coating

| | |
|---|---|
| Ethylcellulose 7 cps | 15 |
| EUDRAGIT RL PO | 15 |
| Triethyl citrate | 3 |
| Isopropyl alcohol-dichloromethane (1:1 | 300 |
| by volume)* | |

*Evaporates during processing

EMCOMPRESS™ is supplied by JRS Products.

Manufacturing process: similar to that of Example 3, except that lactose monohydrate was replaced by dibasic calcium phosphate.

### EXAMPLE 6

### Levetiracetam 500 mg Extended Release Tablets with Water-insoluble Hydrophobic Waxy Excipient, Processed by Melt Granulation

6A: Granules.

| Ingredient | Grams |
|---|---|
| Levetiracetam | 50 |
| Cetostearyl alcohol | 15 |
| Microcrystalline cellulose PH102 | 10 |
| Isopropyl alcohol* | 25 |

*Evaporates during processing.

Manufacturing process:

1) Cetostearyl alcohol was heated to about 50° C. to melt.

2) Levetiracetam and microcrystalline cellulose PH102 were sifted through an ASTM #40 mesh sieve.

3) Step 2) materials were granulated using cetostearyl alcohol of step 1). Additionally, isopropyl alcohol was used to granulate the mass.

4) The granules were dried in an oven for 1 hour at 30° C.

5) Dried granules were sifted through an ASTM #30 mesh sieve and were used for subsequent production of controlled release formulations.

6B-6C: Tablets.

Grams

| Ingredient | 6B | 6C |
|---|---|---|
| Granules of 6A | 30 | 30 |
| Microcrystalline | 4.615 | 4.615 |
| cellulose PH102 | | |
| EUDRAGIT RL PO | — | 0.692 |
| EUDRAGIT RS PO | — | 6.231 |
| Talc | 0.3 | 0.3 |
| Magnesium stearate | 0.3 | 0.3 |
| Colloidal silicon dioxide | 0.3 | 0.3 |

Manufacturing process:

1) Microcrystalline cellulose PH102, EUDRAGIT RL PO, and EUDRAGIT RS PO, as required, were sifted through an ASTM #40 mesh sieve.

2) Granules of 6A were blended with step 1) materials for about 10 minutes.

3) Talc, colloidal silicon dioxide and magnesium stearate were sifted through an ASTM #60 mesh sieve.

4) Step 2) blend was blended with step 3) materials for about 5 minutes.

5) The blend of step 4) was compressed into tablets having 12 to 15 Kp hardness.

We claim:

1. A pharmaceutical formulation, comprising:
a) an inner solid phase comprising levetiracetam or a derivative thereof and at least one hydrophobic material, lipophilic material, or a combination thereof; and, optionally
b) an outer continuous phase comprising at least one hydrophobic material, lipophilic material, or a combination thereof.

2. The pharmaceutical formulation of claim 1, wherein an inner solid phase is a tablet, a minitablet, pellets, beads, or granules.

3. The pharmaceutical formulation of claim 1, wherein a weight ratio of levetiracetam to a total of hydrophobic material and lipophilic material is about 1:0.001 to about 1:10.

4. The pharmaceutical formulation of claim 1, wherein a hydrophobic material comprises an ethylcellulose, a hydroxyethyl cellulose, a water insoluble swellable or nonswellable filler, an ammoniomethacrylate copolymer, a methacrylic acid copolymer, a methacrylic acid-acrylic acid ethyl ester copolymer, a methacrylic acid esters neutral copolymer, a dimethylaminoethyl methacrylate-methacrylic acid esters copolymer, a vinylmethyl ether/maleic anhydride copolymer, a salt or ester thereof, or any mixtures thereof.

5. The pharmaceutical formulation of claim 1, wherein a hydrophobic material comprises an ethylcellulose, an ammoniomethacrylate copolymer, a methacrylic acid copolymer, a methacrylic acid-acrylic acid ethyl ester copolymer, a methacrylic acid esters neutral copolymer, or a dimethylaminoethylmethacrylate-methacrylic acid esters copolymer.

6. The pharmaceutical formulation of claim 1, wherein a lipophilic material comprises a wax, a fatty alcohol, a fatty acid ester, a hydrogenated castor oil, a cottonseed oil, or any mixtures thereof.

7. The pharmaceutical formulation of claim 1, wherein the concentration of hydrophobic material and lipophilic material is in the range of about 1 percent to about 50 percent, or about 1 percent to about 25 percent, by weight.

8. The pharmaceutical formulation of claim 1, providing an extended release of levetiracetam for once-daily administration.

9. The pharmaceutical formulation of claim 1, having a dissolution profile, when tested according to the USP procedure in USP type I or type II apparatus with 100 rpm stirring in purified water at 37° C., wherein:
a) less than about 50 percent of contained levetiracetam is released within about the first 2 hours;
b) less than about 75 percent of contained levetiracetam is released within about the first 4 hours; and
c) more than about 80 percent of contained levetiracetam is released within about the first 12 hours.

10. A pharmaceutical tablet comprising, by weight: about 10 percent to about 75 percent of levetiracetam; and from about 1 percent to about 50 percent of a hydrophobic material, a lipophilic material, or a combination thereof.

11. The pharmaceutical tablet of claim 10, wherein a coating contains a portion of the hydrophobic material, lipophilic material, or combination thereof.

12. The pharmaceutical tablet of claim 10, wherein a hydrophobic material, a lipophilic material, or a combination thereof comprises from about 1 percent to about 25 percent, by weight.

13. The pharmaceutical tablet of claim 12, wherein a coating contains a portion of the hydrophobic material, lipophilic material, or combination thereof.

14. The pharmaceutical tablet of claim 10, wherein a hydrophobic material comprises an ethylcellulose, a hydroxyethyl cellulose, a water insoluble swellable or nonswellable filler, an ammoniomethacrylate copolymer, a methacrylic acid copolymer, a methacrylic acid-acrylic acid ethyl ester copolymer, a methacrylic acid esters neutral copolymer, a dimethylaminoethyl methacrylate-methacrylic acid esters copolymer, a vinylmethyl ether/maleic anhydride copolymer, a salt or ester thereof, or any mixtures thereof.

15. The pharmaceutical tablet of claim 10, wherein a lipophilic material comprises a wax, a fatty alcohol, a fatty acid ester, a hydrogenated castor oil, a cottonseed oil, or any mixtures thereof.

16. The pharmaceutical tablet of claim 10, providing an extended release of levetiracetam for once-daily administration.

17. The pharmaceutical tablet of claim 10, having a dissolution profile, when tested according to the USP procedure in USP apparatus 1 or 2, with 100 rpm stirring, in purified water at 37° C., wherein:
a) less than about 50 percent of contained levetiracetam is released within about the first 2 hours;
b) less than about 75 percent of contained levetiracetam is released within about the first 4 hours; and
c) more than about 80 percent of contained levetiracetam is released within about the first 12 hours.
